(19) United States
(12) Patent Application Publication
(10) Pub. No.: US 2025/0134683 A1
(43) Pub. Date: May 1, 2025

(54) MICRO-NANO MEDICAL STENT FOR DIRECTIONAL, QUANTITATIVE AND TIMED RELEASE OF DRUG AND PREPARATION METHOD THEREOF

(71) Applicant: GUANGDONG UNIVERSITY OF TECHNOLOGY, Guangzhou (CN)

(72) Inventors: Xun CHEN, Guangzhou (CN); Rongguang ZHANG, Guangzhou (CN); Xuanzhi ZHANG, Guangzhou (CN); Xin CHEN, Guangzhou (CN); Han WANG, Guangzhou (CN); Jingsong YAO, Guangzhou (CN)

(73) Assignee: GUANGDONG UNIVERSITY OF TECHNOLOGY, Guangzhou (CN)

(21) Appl. No.: 18/621,119

(22) Filed: Mar. 29, 2024

(30) Foreign Application Priority Data
Oct. 26, 2023 (CN) 202311398653.5

(51) Int. Cl. A61F 2/92; A61K 9/00

(52) U.S. Cl. CPC A61F 2/92; A61K 9/0002

(57) ABSTRACT

The present disclosure belongs to the technical field of drug delivery and biomimetic structures inducing cell proliferation and differentiation, and in particular relates to a micro-nano medical stent for directional, quantitative and timed controlled release of drug and a preparation method thereof. The micro-nano medical stent provided by the present disclosure is composed of an ordered fiber structure electrospun as a skeleton layer and a disordered fiber structure electrospun as an ECM layer that can both load drugs. The micro-nano medical stent can achieve directional, quantitative and timed release of drugs.

### CROSS REFERENCE OF RELATED APPLICATIONS

This application claims priority to Chinese Patent Application No. 202311398653.5, titled “MICRO-NANO MEDICAL STENT FOR DIRECTIONAL, QUANTITATIVE AND TIMED RELEASE OF DRUG AND PREPARATION METHOD THEREOF”, filed on Oct. 26, 2023 with the China National Intellectual Property Administration, which is incorporated herein by reference in its entirety.

### FIELD

The present disclosure belongs to the technical field of drug delivery, and in particular relates to a micro-nano medical stent for directional, quantitative and timed release of drug and a preparation method thereof.

### BACKGROUND

Drug delivery system is a technology that a drug is encapsulated in a carrier so that the drug can better reach the medication site. It can improve the bioavailability, targeting and safety of drugs and achieve efficient drug treatment.

Electrospinning technology is a micro-nano additive technology, which usually consists of a syringe, a high-voltage DC power supply, a conductive collection plate, and a spinning solution or a spinning melt. By achieving jet ejection and drawing through applying a high-voltage electric field on the syringe filled with a spinning solution or a spinning melt and a conductive collection plate, the collection plate can obtain disordered or ordered nanofiber films. The spinning solution or spinning melt can be doped with drugs, so that the nanofiber films can be used as a drug carrier to deliver the drug to the medication site. However, conventional disordered electrospinning technology cannot achieve controllable release of loaded drugs, that is, timed release; moreover, disordered fibers cannot provide effective physical structure guidance signals for cell proliferation and attachment, that is, directional release.

Therefore, we combine coaxial electrospinning technology and near-field direct writing technology, and in response to the demand for directional, quantitative, and timed release of drug-loaded medical micro-nano stents, we propose a near-field direct writing ordered drug-loadable fiber as a skeleton layer to provide a topological structure signal to induce cell proliferation and attachment, and use a coaxial electrospinning technology as an ECM layer to provide a microenvironment for cell proliferation, and achieve controllable sequential release of biochemical signals. Finally, a micro-nano medical stent with directional, quantitative and timed controlled release of drugs and a preparation method thereof are provided, thereby solving the technical problem in the prior art that the nanofiber stent prepared by the electrospinning process cannot achieve directional, quantitative and timed release of drugs.

### SUMMARY

In view of this, the present disclosure provides a micro-nano medical stent for directional, quantitative and timed release of drug and a preparation method thereof, which is used to solve the problem in the prior art that nanofiber stents prepared by electrospinning technology cannot achieve directional, quantitative and timed release of drugs.

The first aspect of the present disclosure provides a micro-nano medical stent for directional, quantitative and timed release of drug, comprising a drug-loadable micron ordered electrospun fiber structure as a skeleton layer and a drug-loadable core-shell nano-disordered electrospun fiber structure as an ECM (extracellular matrix) layer;
wherein, at least one layer of the drug-loadable micron ordered electrospun fiber structure as a skeleton layer and at least one layer of the drug-loadable core-shell nano-disordered electrospun fiber structure as an ECM layer are staggered stacked to form a multi-layer composite electrospun micro-nano medical stent.

Preferably, the micron ordered electrospun fiber structure includes, but is not limited to, an array square ordered electrospun fiber structure or an equally spaced sinusoidal ordered electrospun fiber structure.

Preferably, a single square fiber in the array square fiber structure has a diameter of 3.5-6.5 microns, and the square has a side length of 100-400 microns.

Preferably, a single sinusoid in the equally spaced sinusoidal fiber structure has a meandering angle of 0 to 30°, a spacing of 100 to 400 microns, and a diameter of 3.5 to 6.5 microns.

Preferably, the fiber in the core-shell nano-disordered electrospun fiber structure has a diameter of 100 to 400 nanometers.

Preferably, the composite electrospun layer comprises one layer of the drug-loadable micron ordered electrospun fiber structure as a skeleton layer and one layer of the drug-loadable core-shell nano-disordered electrospun fiber structure as an ECM layer, which are staggered stacked to form a composite electrospun micro-nano medical stent;
or one layer of the drug-loadable micron ordered electrospun fiber structure as a skeleton structure and two layers of the drug-loadable core-shell nano-disordered electrospun fiber structure as an ECM layer, which are staggered stacked to form a composite electrospun micro-nano medical stent;
or two layers of the drug-loadable micron ordered electrospun fiber structure as a skeleton layer and one layer of the drug-loadable core-shell nano-disordered electrospun fiber structure as an ECM layer, which are staggered stacked to form a composite electrospun micro-nano medical stent;
or two layers of the drug-loadable micron ordered electrospun fiber structure as a skeleton layer and two layers of the drug-loadable core-shell nano-disordered electrospun fiber structure as an ECM layer, which are staggered stacked to form a composite electrospun micro-nano medical stent.

Preferably, the drug is roxithromycin and/or human epidermal growth factor gel.

The second aspect of the present disclosure provides a method of preparing the micro-nano medical stent for directional, quantitative and timed release of drug, comprising steps of:
step A: heating and melting a drug-containing polycaprolactone solid, connecting the same to the syringe of a near-field direct writing electrospinning system, and performing ordered near-field direct writing electrospinning to obtain a drug-loaded micron ordered electrospun fiber structure;
step B: repeating step A at least 0 times;
step C: connecting a polylactic acid spinning solution containing a shell layer drug and a polyvinyl alcohol spinning solution containing a core layer drug to the shell layer syringe and core layer syringe of a far-field electrospinning system, respectively, performing disordered far-field electrospinning, and collecting on the surface of the drug-loadable ordered micron fiber structure to obtain a drug-loadable micron ordered electrospun fiber structure as a skeleton layer and a drug-loaded core-shell nano disordered electrospun fiber structure, which are stacked;
step D: repeating step C at least 0 times;
step E: repeating steps A to D.

Preferably, in step A, performing ordered near-field direct writing electrospinning to obtain a drug-loadable micron ordered electrospun fiber structure specifically comprises:
step A1: drawing an ordered fiber trajectory path through CAD drawing software, and then importing the path into an ordered near-field direct writing electrospinning system;
step A2: performing ordered near-field direct writing electrospinning to obtain a drug-loadable micron ordered electrospun fiber structure;

wherein, the ordered fiber trajectory path includes, but is not limited to, an array square ordered fiber trajectory path and an equally spaced sinusoidal ordered fiber trajectory path.

Preferably, in step B, step A is repeated 0 times, and in step D, step C is repeated 0 times to obtain one layer of the drug-loadable micron ordered electrospun fiber structure as a skeleton layer and one layer of the drug-loadable core-shell nano disordered electrospun fiber structure as an ECM layer, which are staggered stacked to form a composite electrospun micro-nano medical stent.

Preferably, in step B, step A is repeated once, and in step D, step C is repeated 0 times to obtain two layers of the drug-loadable micron ordered electrospun fiber structure as a skeleton layer and one layer of the drug-loadable core-shell nano disordered electrospun fiber structure as an ECM layer, which are staggered stacked to form a composite electrospun micro-nano medical stent.

Preferably, in step B, step A is repeated 0 times, and in step D, step C is repeated once to obtain one layer of the drug-loadable micron ordered electrospun fiber structure as a skeleton layer and two layers of the drug-loadable core-shell nano disordered electrospun fiber structure as an ECM layer, which are staggered stacked to form a composite electrospun micro-nano medical stent.

Preferably, in step B, step A is repeated once, and in step D, step C is repeated once to obtain two layers of the drug-loadable micron ordered electrospun fiber structure as a skeleton layer and two layers of the drug-loadable core-shell nano disordered electrospun fiber structure as an ECM layer, which are staggered stacked to form a composite electrospun micro-nano medical stent.

Preferably, the number of the staggered stacking is 2-5 layers.

Preferably, in step C, the polylactic acid spinning solution containing a shell layer drug is doped with a hydrophilic material.

Preferably, the hydrophilic material is polyethylene glycol.

Preferably, the hydrophilic material is added at an amount of 0˜15 wt %.

In summary, the present disclosure provides a micro-nano medical stent for directional, quantitative and timed release of drug and a preparation method thereof. The micro-nano medical stent comprises a drug-loadable micron ordered electrospun fiber structure as a skeleton layer and a drug-loadable core-shell nano disordered electrospun fiber structure, where at least one layer of the drug-loadable micron ordered fiber structure and at least one layer of the drug-loadable core-shell disordered fiber structure are alternately stacked to form a composite electrospun layer.

The micron ordered fiber structure in the composite electrospun layer can guide cells to adhere in a directional manner, and after the micron ordered fiber structure guides cells to adhere, the fiber degrades to achieve the directional release of the loaded drugs to the cells. Moreover, the shell layer electrospun fiber in the core-shell nano disordered electrospun fiber structure degrades first, causing the drugs in the shell layer to be released first. The core layer electrospun fibers degrades later, resulting in the subsequent release of the loaded drugs in the core layer. The control of the degradation sequence of the shell layer electrospun fibers and the core layer electrospun fibers realizes the timed release of drug. Moreover, the drug and the spinning solution are pre-blended, so that the amount of loaded drug can be determined in advance, achieving the quantitative release of drug. Besides, the core-shell nano-disordered electrospun fiber structure is nanometer-sized, and has the characteristics of imitating the cytoplasmic matrix in micron-sized array square ordered fiber structures, which promotes the directional adhesion of cells to the micron ordered fiber structure, thus solving the problem in the prior art that nanofiber stents prepared by electrospinning technology cannot achieve directional, quantitative and timed release of drugs.

The present disclosure belongs to the technical field of drug delivery and biomimetic structures inducing cell proliferation and differentiation, and in particular relates to a micro-nano medical stent for directional, quantitative and timed controlled release of drug and a preparation method thereof. The micro-nano medical stent provided by the present disclosure is composed of an ordered fiber structure electrospun as a skeleton layer and a disordered fiber structure electrospun as an ECM layer that can both load drugs. The skeleton layer can be constructed according to specific applications or be constructed to a specific three-dimensional bionic tissue structure according to corresponding bionic indicators, or be constructed to a specific structure for cell directional adhesion according to a required bionic indicator. The ECM layer facilitate the construction of a bionic microenvironment for cell proliferation; and the fibers in the skeleton layer and ECM layer are core-shell structural fibers, which jointly provide structural topology signal for cell proliferation and differentiation and the improved controlled release biological/chemical signal of the loaded drug. At the same time, controlling the amount of blending of the drug and the spinning solution in advance can achieve quantitative release of the drug. The successive dissolution of shell fibers and core fibers of the core-shell structure achieves the timed release of the drug. The composite fiber layer formed by at least one ordered fiber structure electrospun skeleton layer and at least one layer of disordered nanofiber structure electrospun ECM staggered and stacked can further construct a multi-layered micro-nano medical stent, thereby solving the technical problem in the prior art that the nanofiber stent prepared by the electrospinning process cannot achieve directional, quantitative and timed release of drugs.

### BRIEF DESCRIPTION OF DRAWINGS

In order to more clearly explain the specific embodiments of the present disclosure or the technical solutions in the prior art, the drawings used in the specific embodiments of the description or the prior art will be briefly introduced below. Apparently, the drawings in the following description are some embodiments of the present disclosure. For those of ordinary skill in the art, other drawings can be obtained based on these drawings without exerting inventive efforts.

FIG. 1 is a schematic diagram of the directional adhesion test results of the micro-nano medical stents for directional, quantitative and timed release of drug provided in Examples 1-3 of the present disclosure;

FIG. 2 is a schematic diagram of the directional adhesion osteogenesis test results of the micro-nano medical stents for directional, quantitative and timed release of drug provided in Examples 1-3 of the present disclosure;

FIG. 3 is a scanning electron microscope image of the core-shell structure nanofibers obtained by far-field electrospinning in the drug-loadable platform provided in Example 1 of the present disclosure;

FIG. 4 is a schematic structural diagram of the drug-loadable platform provided for Example 1 of the present disclosure;

FIG. 5 is a scanning electron microscope image of the drug-loadable platform provided for Example 4 of the present disclosure;

FIG. 6 is a schematic diagram of the timed release speed test of the micro-nano medical stents for directional, quantitative and timed release of drug provided in Example 4 and Example 1 of the present disclosure;

FIG. 7 is a schematic diagram of the hydrophilicity test of five types of electrospun fibers provided in Example 4 of the present disclosure;

FIG. 8 is a schematic diagram of the fiber trajectory path during electrospinning in Example 5 of the present disclosure;

FIG. 9 is a schematic diagram of the equally spaced sinusoidal fiber structure electrospinning layer with meandering angles of 0°, 10°, 20° and 30° prepared by electrospinning in Example 5 of the present disclosure;

FIG. 10 is a schematic diagram of the results of the directional adhesion test on the equally spaced sinusoidal fiber structure electrospinning layer with meandering angles of 0°, 10°, 20° and 30°.

### DETAILED DESCRIPTION

The present disclosure provides a micro-nano medical stent for directional, quantitative and timed release of drug and a preparation method thereof, which is used to solve the technical problem in the prior art that the nanofiber stents prepared by the electrospinning process cannot achieve directional, quantitative and timed release of drug.

The technical solution of the present disclosure will be clearly and completely described below with reference to the accompanying drawings. Apparently, the described examples are some of the embodiments of the present disclosure, rather than all of the embodiments. Based on the examples in the present disclosure, all other embodiments obtained by those of ordinary skill in the art without making inventive efforts fall within the protection scope of the present disclosure.

### Example 1

In view of the shortcoming in the prior art that the prepared nanofiber stents cannot achieve directional, quantitative and timed release of drugs in the spatiotemporal dimension,

Example 1 of the present disclosure provides a method of preparing a micro-nano medical stent for directional, quantitative and timed release of drug. The preparation method comprised steps of preparing a spinning substrate and electrospinning;
wherein, the step of preparing a spinning substrate comprised preparing a spinning substrate of a near-field direct writing electrospinning system and preparing a spinning substrate of a far-field electrospinning system;
wherein, preparing a spinning substrate of a near-field direct writing electrospinning system comprised dissolving roxithromycin and polycaprolactone in an organic solvent and then volatilizing the organic solvent through ventilation to obtain a polycaprolactone solid containing roxithromycin, wherein roxithromycin was added at an amount of 5 wt % in the polycaprolactone solid containing roxithromycin;
preparing a spinning substrate of a far-field electrospinning system comprised adding human epidermal growth factor gel to 10 ml of 2 wt % PVA polyvinyl alcohol solution at an amount of 10% of the volume of the PVA polyvinyl alcohol solution and stirring evenly to obtain a PVA polyvinyl alcohol spinning solution containing human epidermal growth factor gel, which was a core layer spinning solution;
preparing a PLA polylactic acid spinning solution containing roxithromycin comprised adding roxithromycin to 10 ml of 2 wt % PLA polylactic acid solution at an amount of 5 wt % and stirring evenly to obtain a PLA polylactic acid spinning solution containing roxithromycin, which was a shell layer spinning solution.

The step of electrospinning comprised preparing through electrospinning an array square ordered electrospun fiber structure and a core-shell nano-disordered electrospun fiber structure, and repeating the same.
Step 1: preparing through electrospinning an array square ordered electrospun fiber structure comprised adding the prepared polycaprolactone solid containing roxithromycin into the first syringe of the near-field direct writing electrospinning system, connecting the syringe to the shell layer spinneret, then setting the operating parameters and environmental parameters of the near-field direct writing electrospinning system, drawing the array square fiber trajectory path needed to be printed by using CAD software, and then importing the path into the near-field direct writing system; starting ordered near-field direct writing spinning, and melt-printing a drug-loadable array square ordered electrospun fiber structure with a specific trajectory, which was labeled as P-100 ordered electrospun fiber structure, wherein a single square fiber in the array square ordered electrospun fiber structure has a side length of 100 μm and a diameter of about 5 μm.
Step 2: preparing through electrospinning a core-shell nano-disordered electrospun fiber structure comprised adding the prepared PLA polylactic acid spinning solution containing roxithromycin to the shell layer syringe of the far-field electrospinning system, connecting the syringe to the shell layer spinneret, adding the prepared PVA polyvinyl alcohol spinning solution containing human epidermal growth factor gel to the core layer syringe of the far-field electrospinning system, connecting the syringe to the core layer spinneret, then setting the operating parameters and environmental parameters of the far-field electrospinning system, starting far-field coaxial electrospinning, printing a drug-loadable disordered electrospun fiber structure, and collecting on the ordered electrospun fiber structure to obtain the stacked ordered electrospun fiber structure and core-shell nano-disordered electrospun fiber structure.
Step 3 comprised repeating electrospinning to prepare an array square ordered electrospun fiber structure and a core-shell nano-disordered electrospun fiber structure to obtain at least one layer of the drug-loadable micron ordered fiber structure and at least one layer of the drug-loadable core-shell disordered fiber structure, which are staggered stacked to form a composite electrospun layer, as a micro-nano medical stent for directional, quantitative and timed release of drug.

It should be noted that steps 1 and 2 can be repeated 0, 1 or 2 times as needed to obtain one layer of drug-loadable micron ordered fiber structure and at least one layer of the drug-loadable core-shell disordered fiber structure, which are staggered stacked, two layers of drug-loadable micron ordered fiber structures and at least two layers of the drug-loadable core-shell disordered fiber structure, which are staggered stacked, or three layers of drug-loadable micron ordered fiber structure and at least three layers of the drug-loadable core-shell disordered fiber structure, which are staggered stacked. The present patent application does not limit the number of repetitions of step 1 and step 2.

It should be noted that, as needed, according to the structural requirements of different layers, in addition to the aligned staggered stacking method used in this example to prepare the drug-loadable micron ordered electrospun fiber structure, non-aligned staggered stacking method can also be used between multiple layers to perform heterogeneous stacking.

### Example 2

Example 2 of the present disclosure provides a method of preparing an array square ordered electrospun fiber structure. The method differs from that of Example 1 in that the fiber trajectory path needed to be printed by the CAD drawing software was changed. The prepared array square fiber structure electrospun layer was used as a micro-nano medical stent for directional, quantitative and timed release of drug. A single square fiber in the micro-nano medical stent for directional, quantitative and timed release of drug had a side length of 200 microns and a diameter of about 5 microns. An array square ordered electrospun fiber structure labeled P-200 was obtained.

### Example 3

Example 3 of the present disclosure provides a method of preparing an array square ordered electrospun fiber structure. The method differs from that of Example 1 in that the fiber trajectory path needed to be printed by the CAD drawing software was changed. The prepared array square fiber structure electrospun layer was used as a micro-nano medical stent for directional, quantitative and timed release of drug. A single square fiber in the micro-nano medical stent for directional, quantitative and timed release of drug had a side length of 400 microns and a diameter of about 5 microns. An array square ordered electrospun fiber structure labeled P-400 was obtained.

### Experimental Example 1

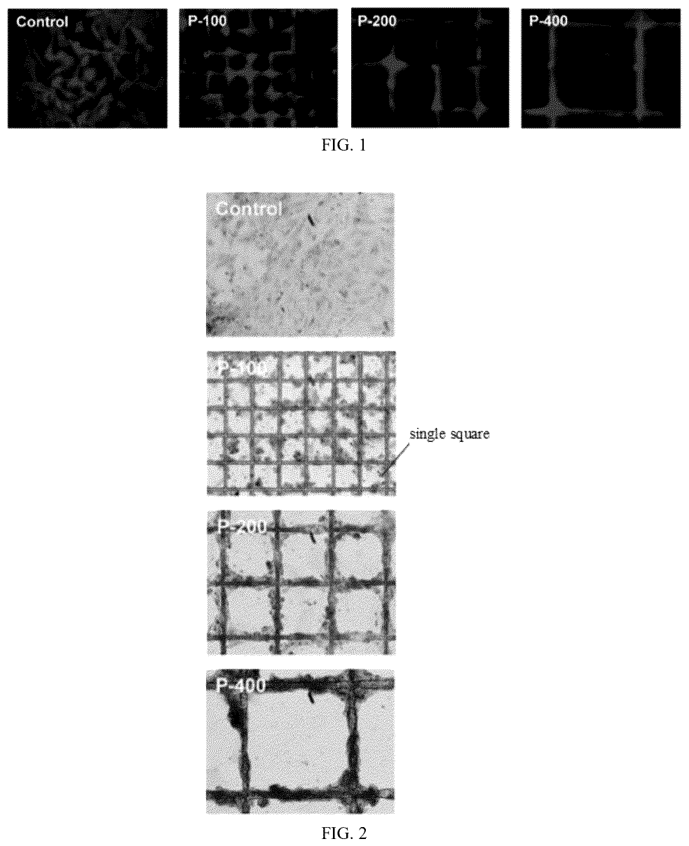

In this experimental example 1, the array square ordered electrospun fiber structures prepared in Examples 1-3 were subjected to measurements of cell directional adhesion and release of drug. The measurement results are shown in FIGS. 1-2. In FIG.s 1-2, Control, P-100, P-200 and P-400 respectively represent the adhesion of cells on stent-free conductive glass and on array square fiber structure electrospun layers with a diameter of about 5 microns and a single square fiber side length of 100, 200 and 400 μm.

Among them, the measurements of cell directional adhesion and release of drug were performed using Saos-2 human osteosarcoma cells adhering to stent-free conductive glass as the control group (Control). The experimental results are shown in FIG. 1. It can be seen from FIG. 1 that compared with the adhesion effect of cells on stent-free conductive glass in the control group (Control), the array square ordered electrospun fiber structure prepared in the examples of the present disclosure can show a strong guiding effect on cell orientation and can achieve directional adhesion of cells, indicating that the array square ordered electrospun fiber structure suitable for cell adhesion provided by the patent application, which is printed using near-field direct writing system after pre-drawing the electrospinning fiber path by CAD drawing software, is conducive to cell adhesion and directional release of drug. As can be seen from FIG. 1, when the fiber spacing/side length of a single square fiber is 100 μm, the adhesion effect is better.

Further, the osteogenic activity of Saos-2 cells in micro-nano medical stents with various directional, quantitative and timed release of drug is shown in FIG. 2, i.e. ARS staining image on day 14. The formation of mineralized nodules is an important indicator of calcium mineralization, which was confirmed in this study by ARS staining 14 days after osteogenic induction. ARS staining showed that the calcium nodules formed on P-100, P-200 and P-400 were mainly attached to the array square ordered electrospun fiber structure, showing significant directional cell guidance.

The above shows that the micron-ordered fiber structure in the composite electrospun layer provided in Example 1 can guide cell adhesion in a directional manner. After the micron-ordered fiber structure guides the cells, the fibers degrade to achieve the directional release of the loaded drugs to the cells. The core-shell nano-disordered electrospun fiber structure in the composite electrospun layer is nano-sized, and has the characteristics of imitating the cytoplasmic substrate in micron-sized array square ordered fiber structure, which promotes the directional adhesion of cells to the micron-ordered fiber structure, and facilitates the directional release of drugs into cells.

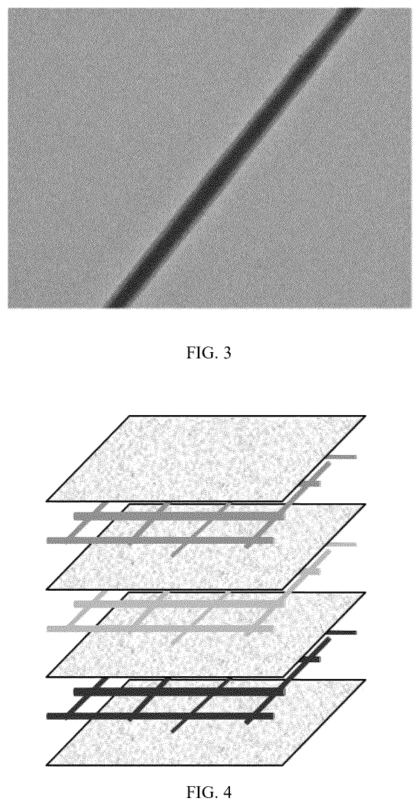

Moreover, the transmission electron microscopic image of the core-shell nano-disordered electrospun fiber structure in the composite electrospun layer prepared in Example 1 is shown in FIG. 3. The core-shell nano-disordered electrospun fiber had a diameter of about 200 nanometers, comprising core layer fiber and shell layer fiber. When the core-shell nano-disordered electrospun fiber structure in the composite electrospun layer prepared in the examples of the present disclosure was used for release of drug, roxithromycin loaded in the shell layer fiber in the electrospun layer was released in advance, and the human epidermal growth factor in the core layer fiber was subsequently released. The coaxial electrospinning realizes the sequential and timed release of the two drugs in the same electrospun layer. Further, the electrospun layer can be prepared by triaxial electrospinning or multiaxial electrospinning process, which has a core-shell structure of three core layers or multi-core layers, thereby achieving the sequential and timed release of three or more drugs in the same electrospun layer.

In addition, the roxithromycin and human epidermal growth factor in the composite electrospun layer of the present disclosure can be controlled. The roxithromycin and human epidermal growth factor can be pre-added to the spinning solution such as PVA polyvinyl alcohol at an amount needed to be released, thereby realizing quantitative release of drugs, so that the composite electrospun layer provided by the present patent application achieves directional, quantitative and timed release of drug in the spatiotemporal dimension.

### Example 4

Example 4 of the present disclosure provides a method of preparing a micro-nano medical stent for directional, quantitative and timed release of drug. The purpose is to provide a drug-loadable platform that further achieves speed control of timed release on the basis of directional, quantitative and timed release. The method differs from that of Example 1 in that 5 wt %, 10 wt % and 15 wt % polyethylene glycol were added to the PLA polylactic acid spinning solution containing roxithromycin.

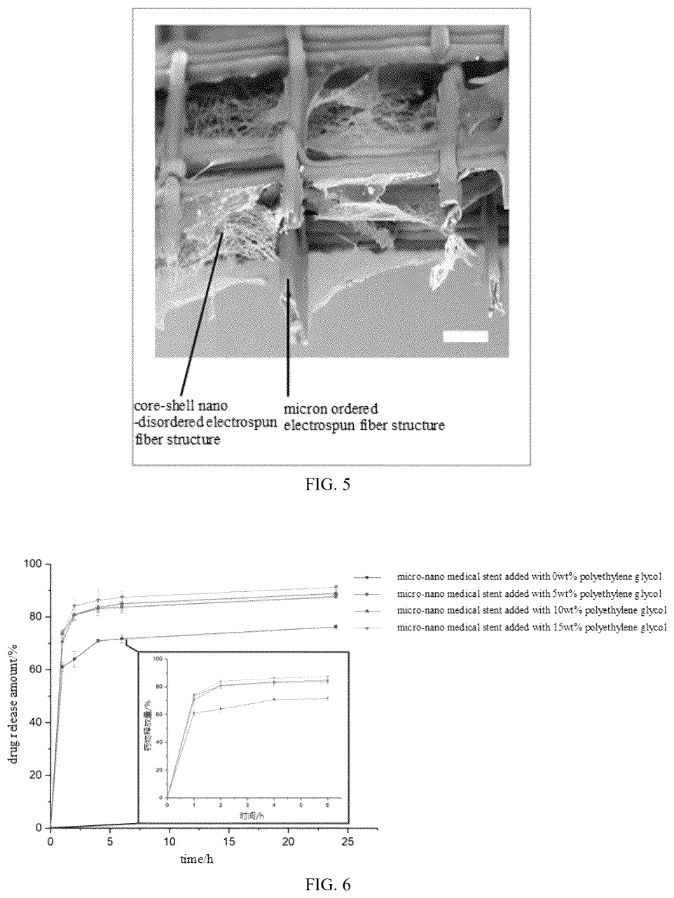

The step of electrospinning comprised preparing through electrospinning an array square ordered electrospun fiber structure and a core-shell nano-disordered electrospun fiber structure, and repeating the same.
Step 1: preparing through electrospinning an array square ordered electrospun fiber structure comprised adding the prepared polycaprolactone solid containing roxithromycin into the first syringe of the near-field direct writing electrospinning system, connecting the syringe to the shell layer spinneret, then setting the operating parameters and environmental parameters of the near-field direct writing electrospinning system, drawing the array square fiber trajectory path needed to be printed by using CAD software, and then importing the path into the near-field direct writing system; starting ordered near-field direct writing spinning, and printing a drug-loadable array square ordered electrospun fiber structure with a specific trajectory, which was labeled as P-100 ordered electrospun fiber structure, wherein a single square fiber in the array square ordered electrospun fiber structure had a side length of 100 μm and a diameter of about 5 μm.
Step 2: preparing through electrospinning a core-shell nano-disordered electrospun fiber structure comprised adding the prepared PLA polylactic acid spinning solution containing roxithromycin to the shell layer syringe of the far-field electrospinning system, connecting the syringe to the shell layer spinneret, adding the prepared PVA polyvinyl alcohol spinning solution containing human epidermal growth factor gel to the core layer syringe of the far-field electrospinning system, connecting the syringe to the core layer spinneret, then setting the operating parameters and environmental parameters of the far-field electrospinning system, starting far-field coaxial electrospinning, printing a drug-loadable disordered electrospun fiber structure, and collecting on the ordered electrospun fiber structure to obtain the stacked ordered electrospun fiber structure and core-shell nano-disordered electrospun fiber structure; wherein, polyethylene glycol was added to the shell layer nano-disordered electrospun fiber in the core-shell nano-disordered electrospun fiber structure at an amount of 5 wt %, 10 wt % and 15 wt %.
Step 3 comprised repeating electrospinning to prepare an array square ordered electrospun fiber structure and a core-shell nano-disordered electrospun fiber structure to obtain two layers of the drug-loadable micron ordered fiber structure and two layers of the drug-loadable core-shell disordered fiber structure, which were staggered stacked to form a composite electrospun layer, as a micro-nano medical stent for directional, quantitative and timed release of drug. The scanning electron microscope is shown in FIG. 5.

The micro-nano medical stents prepared for directional, quantitative and timed release of drug in Example 4 and Example 1 were subjected to timed release rate analysis. The analysis results are shown in FIG. 6. It can be seen from FIG. 6 that as the amount of polyethylene glycol added increased, the timed release rate of roxithromycin in the drug-loadable platform also increased, that is, the release rate of roxithromycin gradually slowd down from the outside to the inside, achieving a sustained release effect, making the medicinal effect both efficient and durable, thereby improving the therapeutic effect. In addition, drugs that need to be released quickly can be loaded into spinning fibers with high content of polyethylene glycol as needed. Drugs that need to be released slowly can be loaded into spinning fibers with no or less polyethylene glycol added. The micro-nano medical stent for directional, quantitative and timed release of drug provided by the present disclosure can be added with hydrophilic materials such as polyethylene glycol. After adding the hydrophilic material, the timed release rate of the drug in the micro-nano medical stent for directional, quantitative and timed release of drug was controlled. Moreover, in order to change the timed release rate of the drug, the preparation can also be controlled to obtain a micro-nano medical stent for directional, quantitative and timed release of drug comprising a shell layer fiber doped with 0 wt % polyethylene glycol, a first core layer fiber doped with 5 wt % polyethylene glycol, a second core layer fiber doped with 10 wt % polyethylene glycol, and a third core layer fiber doped with 15 wt % polyethylene glycol from the outer to the inner layer.

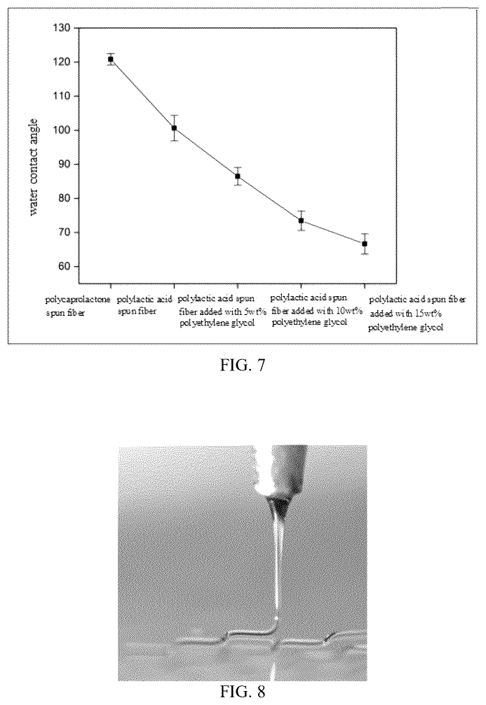

In order to further determine the mechanism by which polyethylene glycol is added to control timed release rate, the shell layer nano-disordered electrospun fibers of the micro-nano medical stents for directional, quantitative and timed release of drug prepared in Examples 4 and 1 and a conventional polycaprolactone-based nano-disordered electrospun fiber were subjected to hydrophilicity test. The test is shown in FIG. 7. It can be seen from FIG. 7 that as the amount of polyethylene glycol added increased, the hydrophilicity of the electrospun fiber also increased, which suggests that the mechanism of the present disclosure achieving drug timed release rate control is to control hydrophilicity, promote the rapid degradation of well-hydrophilic spinning fibers, and slow degradation of poorly hydrophilic spinning fibers, thereby achieving a further control of a timed release rate based on directional, quantitative and timed release.

### Example 5

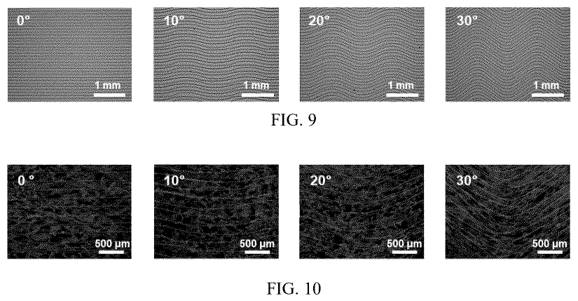

Example 5 of the present disclosure provides a method of preparing a micro-nano medical stent for directional, quantitative and timed release of drug. The method differs from that in Example 1 in that the fiber trajectory path needed to be printed by the CAD drawing software was changed. The fiber trajectory path was an equally spaced sinusoidal curve with a meandering angle of 0°, 10°, 20° and 30°. The fiber trajectory path is shown in FIG. 8. The electrospinning product is an equally spaced sinusoidal fiber structural electrospun layer with a meandering angle of 0˜30°, as shown in FIG. 9, which served as a micro-nano medical stent for directional, quantitative and timed release of drug.

The micro-nano medical stent for directional, quantitative and timed release of drug prepared in Example 5 was analyzed. The directional adhesion test results are shown in FIG. 10. In FIGS. 10, 0°, 10°, 20° and 30° respectively represent cells adhesion to the electrospun layer of sinusoidal fiber structures with equal spacing at meandering angles of 0°, 10°, 20° and 30°. As can be seen from FIG. 1, compared with the equally spaced sinusoidal fiber structure electrospun layer with a meandering angle of 0°, the equally spaced sinusoidal fiber structure electrospun layer with a meandering angle of 30° prepared in the example of the present disclosure showed a strong guiding effect on cell orientation and achieves cell adhesion. This shows that the method provided by the present patent application comprising pre-drawing the electrospun fiber path to be printed using CAD drawing software and then printing electrospun fibers with preset structures suitable for cell adhesion using a near-field direct writing system, is conducive to cell adhesion and directional release of drug.

The above examples are only used to illustrate the technical solutions of the present disclosure, but not to limit them. Although the present disclosure has been described in detail with reference to the foregoing examples, those of ordinary skill in the art should understand that the technical solutions described in the examples can still be modified, or some or all of the technical features can be equivalently replaced; and these modifications or replacements do not cause the essence of the corresponding technical solutions to depart from the scope of the technical solutions in the examples of the present disclosure.

1. A micro-nano medical stent for directional, quantitative and timed release of drug, comprising a drug-loadable micron ordered electrospun fiber structure as a skeleton layer and a drug-loadable core-shell nano-disordered electrospun fiber structure as an ECM (extracellular matrix) layer;
wherein, at least one layer of the drug-loadable micron ordered electrospun fiber structure as a skeleton layer and at least one layer of the drug-loadable core-shell nano-disordered electrospun fiber structure as an ECM layer are staggered stacked to form a multi-layer composite electrospun micro-nano medical stent.

2. The micro-nano medical stent for directional, quantitative and timed release of drug according to claim 1, wherein the drug-loadable micron ordered electrospun fiber structure as a skeleton layer is able to construct a three-dimensional bionic tissue structure according to specific applications or construct a specific structure according to required bionic indicators, including, but not limited to, an array square ordered electrospun fiber structure or an equally spaced sinusoidal ordered electrospun fiber structure.

3. The micro-nano medical stent for directional, quantitative and timed release of drug according to claim 1, wherein the multi-layer composite electrospun micro-nano medical stent comprises one layer of the drug-loadable micron ordered electrospun fiber structure as a skeleton layer and one layer of the drug-loadable core-shell nano-disordered electrospun fiber structure as an ECM layer, which are staggered stacked to form a multi-layer composite electrospun micro-nano medical stent;
or one layer of the drug-loadable micron ordered electrospun fiber structure as a skeleton layer and two layers of the drug-loadable core-shell nano-disordered electrospun fiber structure as an ECM layer, which are staggered stacked to form a composite electrospun micro-nano medical stent;
or two layers of the drug-loadable micron ordered electrospun fiber structure as a skeleton layer and one layer of the drug-loadable core-shell nano-disordered electrospun fiber structure as an ECM layer, which are staggered stacked to form a composite electrospun micro-nano medical stent;
or two layers of the drug-loadable micron ordered electrospun fiber structure as a skeleton layer and two layers of the drug-loadable core-shell nano-disordered electrospun fiber structure as an ECM layer, which are staggered stacked to form a composite electrospun micro-nano medical stent.

4. The micro-nano medical stent for directional, quantitative and timed release of drug according to claim 3, wherein, the stacking form of the drug-loadable micron ordered electrospun fiber structure as a skeleton layer includes aligned staggered stacking or non-aligned staggered stacking, wherein the skeleton layer constructs a three-dimensional bionic tissue structure according to needs by printing the same structure or different structures layer by layer, and connected structures can be established between the layers.

5. A method of preparing the micro-nano medical stent for directional, quantitative and timed release of drug according to claim 1, comprising steps of:
step A: heating and melting a drug-containing polycaprolactone solid, connecting the same to the syringe of a near-field direct writing electrospinning system, and performing ordered near-field direct writing electrospinning to obtain a drug-loaded micron ordered electrospun fiber structure;
step B: connecting a polylactic acid spinning solution containing a shell layer drug and a polyvinyl alcohol spinning solution containing a core layer drug to the shell layer syringe and core layer syringe of a far-field electrospinning system, respectively, performing disordered far-field electrospinning at a temperature, humidity and gas environment required for maintaining activity of loaded drug, and collecting on the surface of the drug-loaded ordered micron fiber structure to obtain a drug-loaded micron ordered electrospun fiber structure as a skeleton layer and a drug-loaded core-shell nano disordered electrospun fiber structure, which are stacked;
step C: alternately repeating step A and B at least 1 time.

6. The method of preparing the micro-nano medical stent for directional, quantitative and timed release of drug according to claim 5, wherein, performing ordered near-field direct writing electrospinning to obtain a drug-loaded micron ordered electrospun fiber structure in step A specifically comprises:
step A1: drawing an ordered fiber trajectory path through CAD drawing software, and then importing the path into an ordered near-field direct writing electrospinning system;
step A2: performing ordered near-field direct writing electrospinning to obtain a drug-loaded micron ordered electrospun fiber structure;
wherein, the ordered fiber trajectory path includes, but is not limited to, an array square fiber trajectory path and an equally spaced sinusoidal fiber trajectory path.

7. The method of preparing the micro-nano medical stent for directional, quantitative and timed release of drug according to claim 5, wherein, in step B, the polylactic acid spinning solution containing a shell layer drug is doped with a hydrophilic material such that the control of degradation rate is realized, and the ECM layer with a specific number of layers can have the same or different degradation rates, thus realizing controllable release sequence of loaded drug at different layers.
